(19) United States
(12) Patent Application Publication
(10) Pub. No.: US 2002/0006953 A1
(43) Pub. Date: Jan. 17, 2002

(54) MODIFICATION OF CHOLESTEROL CONCENTRATIONS WITH CITUS PHYTOCHEMICALS

(76) Inventors: CARLA R. MCGILL, SARASOTA, FL (US); NANCY R. GREEN, BRADENTON, FL (US)

Correspondence Address: COOK ALEX MCFARRON MANZO, CUMMINGS & MEHLER LTD, 200 WEST ADAMS STREET, SUITE 2850, CHICAGO, IL, 60606

(21) Appl. No.: 09/435,304

(22) Filed: Nov. 5, 1999

(51) Int. Cl. A61K 31/47; A61K 31/35; A61K 31/015

(52) U.S. Cl. 514/453; 514/455; 514/764; 426/534

(57) ABSTRACT

Methods, products and compositions are provided which, when administered to a mammal, including humans, raises HDL serum cholesterol levels, while typically also lowering the ratio of LDL to HDL serum cholesterol levels. An effective amount of one or more of a monoterpene, a terpene and a flavonoid are included in the treatment composition.

### BACKGROUND OF THE INVENTION

This invention generally relates to therapeutic compositions and procedures for modifying cholesterol levels in mammals. More particularly, the invention relates to modifying cholesterol levels in a manner which is believed to result in health benefits, especially cardiovascular health benefits. The effects achieved in accordance with the present invention are consistent with reduced risks of cardiovascular disease.

It is generally accepted that an important component in maintaining a profile for good cardiovascular health is the maintenance of desirable cholesterol levels. Currently it is generally accepted that an individual should avoid certain elevated plasma total cholesterol levels. Two major components of plasma cholesterol are low-density lipoprotein (LDL) cholesterol and high-density lipoprotein (HDL) cholesterol. More particularly, LDL cholesterol levels should be maintained below an acceptable level, while high HDL cholesterol levels are considered to contribute cardiovascular health. It is generally accepted that a decreased LDL to HDL cholesterol ratio is an advantageous goal for those whose cholesterol ratio is higher than desirable levels.

A typically accepted dietary intervention regimen for altering blood cholesterol concentrations is to take measures in order to reduce LDL cholesterol levels. Generally, such dietary intervention does not enjoy the ability of increasing HDL cholesterol. It will be appreciated that, if a viable dietary intervention program were available for increasing HDL cholesterol and for decreasing LDL to HDL cholesterol levels, considerable potential benefits would obtain.

Previous epidemiological studies suggested that high dietary intake of fruit and vegetables is associated with reduced risk of coronary heart disease. See, for example, Bors W, Heller W, Michel C, Saran M. Flavonoids as antioxidants: determination of radical scavenging efficiencies. Method Enzymol 1990;186:343-55. Dietary flavonoids have been proposed to exert the cardioprotective action mainly as inhibitors of LDL oxidation and platelet aggregation. Cook N C, Samman S. Flavonoids-chemistry, metabolism, cardioprotective effects, and dietary sources. J Nutr Biochem 1996;7:6676. Some flavonoids, especially those from soybeans, consisting mainly of the isoflavone genistein, have also been suggested to reduce hypercholesterol. Anthony M S, Clarkson T B, Hughes C L Jr., Morgan T M, Burke G L. Soybean isoflavones improve cardiovascular risk factors without affecting the reproductive system of peripubertal Rhesus monkeys. J Nutr 1996;126:43-50.

In addition, numerous studies have demonstrated various beneficial effects of several vitamins that are abundant in fruits and vegetables. The vitamins C, E and beta-carotene have suggested to act mainly as antioxidants. Charleux J L. Beta-carotene, vitamin C, and vitamin E: the protective micronutrients. Nutr Rev 1996;54:S109-S114. Folic acid and natural folate present at high levels in citrus fruit and in many green vegetables also have been shown to reduce plasma levels of homocyst(e)ine, an intermediate in methionine metabolism, implicated as a risk factor in cardiovascular disease. Jacques P F, Selhub J, Bostom A G, Wilson P W F, Rosenberg I H. The effect of folic acid fortification on plasma folate and total homocysteine concentrations. N. Engl J Med 1999;340:1449-54. Brouwer I A, Dusseldorp M V, West C E, Meyboom S, Thomas C M G, Duran M, van het Hof K H, Eskes T K A B, Hautvast J G A J, Steegers-Theunissen R P M. Dietary folate from vegetables and citrus fruit decreases plasma homocysteine concentrations in humans in a dietary controlled trial. J Nutr 1999;129:1135-9.

Dietary citrus juices, especially orange juice and grapefruit juice, are recognized as rich sources of minor components, including phytochemicals such as flavonoids, as well as known essential human nutrients such as folate and vitamin C. High concentrations of folate present in both juices could contribute to their cardioprotective action by reducing plasma homocyst(e)ine, and high concentrations of vitamin C could decrease susceptibility of lipoproteins to oxidation. The present invention recognizes that citrus source phytochemicals play another role in cholesterol health enhancement, whether alone on in combination with one or more of the essential human nutrients.

The effect of citrus juices and their principal flavonoids on cholesterol metabolism has been tested in rabbits, rats and in human liver cell line HepG2. In rabbits with experimental hypercholesterolemia induced by feeding cholesterol-free, casein-based, semipurified diet, replacing drinking water with either orange juice or grapefruit juice (reconstituted from frozen concentrate at twice normal strength) reduced serum LDL cholesterol by 43% and 32%, respectively. Kurowska E M, Borradaile N M, Spense J D, Carroll K K. Hyporcholesterolemic effects of dietary citrus juices in rabbits. Nutr Res 1999. This was associated with a significant, 42% reduction of liver cholesterol esters but not with increases in fecal excretion of cholesterol or bile acids. In addition, dietary supplementation with mixtures of principal citrus flavonoids has been shown to lower serum cholesterol in rats fed a cholesterol-rich diet. Bok S H, Lee S H, Park Y B, Bae K H, Son K H, Jeong T S, Choi M S. Plasma and hepatic cholesterol and hepatic activities of 3-hydroxy-3-methyl-glutaryl-CoA reductase and acyl CoA; cholesterol transferase are lower in rats fed citrus peel extract or a mixture of citrus bioflavonoids. J Nutr 1999;129:1182-5. In this animal model, the 0.5% dietary supplementation with flavonoids also inhibited in vitro activities of HMGCoA reductase and acyl CoA:cholesterol O-acytltransferase (ACAT), two key liver enzymes involved in the regulation of cholesterol metabolism. In HepG2 cells, both hesperetin and naringinin reduced the net secretion of apo B, the protein component of LDL, by inhibiting the synthesis of cellular lipids, especially cholesterol esters. Borradaile N M, Carroll K K, Kurowska E M. Regulation of HepG2 cell apolipoprotein B metabolism by the citrus flavanones hesperetin and naringinin. Lipids 1999;34:591-8. In addition to flavonoids, citrus juices contain relatively high concentrations of limonoids, mostly bitter triterpene derivatives which also demonstrated apo B-lowering potential in HepG2 cells. Kurowska E M, Hasegawa S, and Manners G D (1999). Regulation of apo B production: HepG2 cells by citrus limonoids. In: Berhow E M, Hasegawa S, Manners G D, eds.

Citrus Limonoids

Functional Chemicals in Agriculture and Foods.

ACS Book Series, 1999 (in press).

Limited information is available regarding the potential cardioprotective effects of dietary orange juice in humans. In one recent study conducted in young normocholesterolemic men, intake of orange juice reduced lipoprotein oxidation, presumably due to high content of vitamin C, but did not change plasma lipid profile. Harats D, Chevion S, Nahir M, Norman Y, Sagee O, Berry E M. Citrus fruit supplementation reduces lipoprotein oxidation in young men ingesting a diet high in saturated fat: presumptive evidence for an interaction between vitamins C and E in vivo. Am J Clin Nutr 1998;67:240-5. Another trial, in which unspecified doses of citrus fruit and green vegetables were added to a diet to increase natural dietary folate, showed that this intervention significantly increased plasma content of folate and reduced plasma content of homocyst(e)ine in healthy subjects. Brouwer et al., supra.

### Summary of the Invention

In accordance with the present invention, it is demonstrated that compositions having phytochemicals which are found in dietary citrus sources can produce beneficial changes in plasma lipids and cholesterol concentrations, especially in mildly to moderately hypercholesterolemic subjects. The compositions beneficially change cholesterol levels. More particularly, HDL levels are increased. Also typical of the present invention is a decrease in the LDL to HDL serum cholesterol ratio. The compositions advantageously include one or more of phytochemicals selected from the group consisting monoterpenes, terpenes, and flavonoids. The compositions and their components are present at a dosage level which is effective in increasing HDL serum cholesterol levels and/or in decreasing the LDL to HDL serum cholesterol ratio for the subject being treated.

It is accordingly a general object of the present invention to modify bloodstream cholesterol concentrations through the use of citrus phytochemicals.

Another object of this invention is to provide a composition and method for increasing HDL cholesterol levels in mammals, particularly in humans.

Another object of the present invention is to provide an improved composition and method for decreasing the LDL to HDL cholesterol ratio in the bloodstream of a living being.

Another object of this invention is to provide an improved composition and method for modifying citrus phytochemical combinations for adjusting serum cholesterol levels.

Another object of this invention is to provide improved compositions and methods of their production and use, which compositions incorporate citrus phytochemicals for serum cholesterol modification.

Another object of the present invention is to provide improved serum cholesterol treatment compositions which incorporate combinations of citrus phytochemicals.

Another object of this invention is to provide improved serum cholesterol adjusting treatments which administer citrus phytochemicals in accordance with simultaneous, separate or sequential use.

These and other objects, features and advantages of the present invention will be apparent from and clearly understood through a consideration of the following detailed description.

### BRIEF DESCRIPTION OF THE DRAWINGS

In the course of this description, reference will be made to the attached drawings wherein:

FIG. 1 is a chart of dosing design procedures followed during testing reported herein;

FIG. 2 is a data table laying out analyses of dietary records concerning the Example; and

FIG. 3 is a dietary table setting out changes in characteristics and bloodstream analyses during the Example.

### DESCRIPTION OF THE PREFERRED EMBODIMENTS

Treatment compositions and methods according to the present invention embody the use of phytochemicals of the type which naturally occur within citrus fruits. Included are phytochemicals from different components of citrus fruits, including the juice, juice sacks, pulp, peel, seeds and the like. The phytochemicals in accordance with the invention are the ones which absorb into the bloodstream so as to come into contact with components thereof, such as serum cholesterol The treatment compositions and methods incorporate these phytochemicals at concentrations which achieve the principal effects of the present invention, namely increasing HDL serum cholesterol levels and decreasing LDL to HDL serum cholesterol ratios.

The treatment compositions can be administered in any suitable form such as liquid, solid, injection, infusion, surface application and the like. When in the liquid form, water is a suitable carrier. Carriers for other modes of application are generally appreciated within the relevant arts. An example of a suitable treatment composition having an aqueous carrier can take the form of juices. However constituted, the treatment compositions according to the invention include one or more phytochemicals at the levels providing the beneficial serum cholesterol modifications which are characteristic of the invention.

Citrus juices, for example, are rich sources of relatively minor nutrients. As discussed herein, these minor nutrients are categorized as either known essential human nutrients or phytochemicals. An essential nutrient is vitamin C, known to decrease the susceptibility of lipoproteins to oxidation. Others are folates, believed to effect reduction of plasma homocysteine/homocystine. These so-called minor essential nutrients are understood herein to be excluded from being encompassed by the term phytochemicals as used herein.

Phytochemicals fall within the families generally recognized as monoterpenes, terpenes and/or flavonoids. Examples of monoterpenes include limonene and d-limonene, typically found in peel oil.

The limonoid or limonoid glucoside group of the terpene family includes phytochemicals such as limonin or limonin glucoside from citrus seeds, as well as nomilin. Others within this group of the terpene family are liminol, deoxyliminic acid, limonin carboxymethoxime, limonin-17-O-beta-d-glucoside, obacunone, obacunone-17-O-beta-d-glucoside, nomilin-17-O-beta-d-glucoside, deacetylnomilin, deacetylnomilin-17-O-beta-d-glucoside and deacetylnomilic-17-O-beta-d-glucoside.

Included within the flavanones or flavanone glycosides group of the flavenoid family are the aglycones naringinin and hesperetin, as well as the glucosides naringin and hesperidin, or narirutin. Each of these flavanones is polyphenolic, and each is typically found in citrus peel and juices. Additional flavanones are eriocitrin (typically found in lemon and lime), didymin and poncitrin.

The methoxyflavone group of the flavonoid family also encompasses polyphenolic compounds. These methoxylated flavones include tangeretin and nobiletin. Other methoxyflavones include sinensetin, heptamethoxyflavone, tetra-O-methylscutellarein, and hexa-O-methylgossypetin.

Regarding the serum cholesterol modifying effective amount of the phytochemicals incorporated into the compositions, such will vary depending upon the particular phytochemical. It will be generally understood that the quantity of phytochemical to be administered can be determined by assessing whether or not the particular phytochemical increases HDL serum cholesterol to a significant extent at the particular amount. Amounts or dosage levels can be expressed as a weight percent or on a parts per million basis. Dosages also can be expressed as weight of phytochemical per unit of body weight or blood serum volume. A typical phytochemical dosage can be expressed as, for example, a specified level of milligrams per kilogram of body weight. Alternatively, and as used generally herein, dosages can be expressed as certain quantity of phytochemical or essential human nutrient administered on a daily basis. A typical effective dosage level for a minor nutrient such as vitamin C could be expressed as, for example, 75 mg per day, while that for folate can be expressed as, for example, 63 μg per day, both typically as included in the treatment composition in combination with one or more phytochemicals.

Examples of daily effective dosages of the phytochemicals include the following. In the monoterpenes family, a typical limonene dosage is on the order of at least about 75 mg per day, preferably at least about 100 mg per day. In the terpene family, members of the limonoid glucoside group have a typical dosage level of at least about 75 mg per day, preferably at least about 150 mg per day. Of these, limonin glucoside has a dosage level of at least about 60 mg per day, preferably at least about 100 mg per day. A limonin level can be as low as about 1 mg per day. The flavanone glucoside group of the flavonoid family has a typical dosage level of at least about 100 mg per day. Of specific flavanone glucosides, hesperidin would have a typical treatment dosage of at least about 50 mg per day, and naringin would have a typical treatment dosage level of at least about 5 mg per day. Regarding the methoxyflavone group of the flavonoid family, the dosage level can be as low as about 1 mg per day.

Concerning these dosage levels, the levels can be considered to be either dosage levels of the sole component, or more typically, dosage levels when a multiplicity of these phytochemicals are incorporated within the treatment composition, either alone or in combination with one or more of the essential human nutrients.

The treatment compositions and methods according to the present invention are suitable for use in altering cholesterol levels of mammals, particularly of humans. Basically, the invention administers one or more of a monoterpene, a terpene, or a flavonoid at levels at which the HDL serum cholesterol amount is increased. These levels are generally exemplified hereinabove. The invention is especially effective when the subject being treated has an undesirably low HDL serum cholesterol level and/or undesirably high LDL/HDL serum cholesterol ratio. Treatment times for achieving the cholesterol modifying effect typically will proceed with these dosage levels for several days to a few weeks as an initial effective dosage regimen.

The following Example provides illustrations of the disclosure herein.

### EXAMPLE

Subjects

Twenty-five subjects (16 men and 9 postmenopausal women, average age 55±11 years, average body weight 78±13 kg) were subjected to testing. Most of the participants had moderately elevated initial plasma total and LDL cholesterol concentrations (5.5 to 8.4 mmol/L and 3.3 to 5.1 mmol/L, respectively) while five subjects were mildly hypercholesterolemic or normocholesterolemic (plasma total and LDL cholesterol concentrations of 4.4 to 5.2 mmol/L and 2.4 to 3.1 mmol/L, respectively). Each participant was required to: 1) have initial fasting plasma tricylglycerol concentrations in the normal range (subject range 0.8 to 3.4 mmol/L); 2) be habitual/occasional orange juice drinkers; 3) be free of thyroid disorders, kidney disease and diabetes; 4) have alcohol intake not greater than two drinks per day; and 5) if females, not taking hormone replacement. A small number of the participants had been taking prescribed cholesterol-lowering medication before the study, and these were asked to discontinue the treatment six weeks before the onset of the study. Participants were also advised to 1) follow the American Heart Association (AHA) Step One lipid lowering diet for six weeks before the study and for the duration of the trial; and 2) avoid taking supplements such as vitamins, minerals or flavonoids during the study.

Experimental Protocol

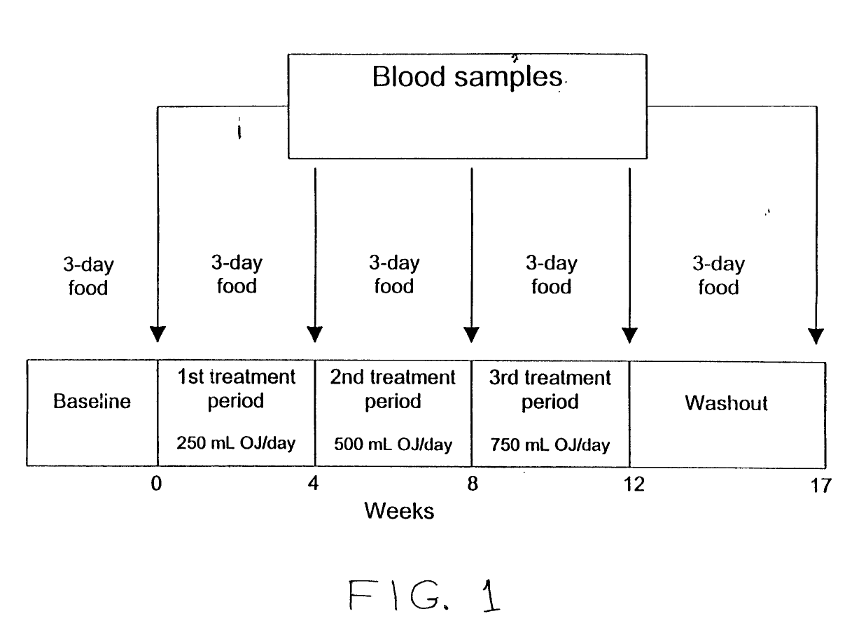

The study was performed according to standard dosing design procedures, the details of which are included in FIG. 1. Participants consumed the AHA Step One diet during the full 17 weeks of the study. During this time, they were asked to incorporate in this diet a treatment formulation of one, two or three glasses, 250 ml each, of orange juice of the not-from-concentrate type per day, sequentially, for three separate four week periods, followed by a five-week washout period. Fasting blood samples were drawn from the anticubital vein of the forearm at the five time points specified in FIG. 1. Plasma liopoproteins (VLDL, LDL and HDL) were separated by discontinues density gradient ultracentrifugation, as described by Redgrave et al. Redgrave T G, Roberts D C K, West C E. Separation of plasma lipoproteins by density gradient ultracentrifugation. Anal Biochem 1975;63:42-9. The concentration of cholesterol and triacylglycerols were measured in a clinical biochemistry laboratory, and the evaluations were done by enzymatic timed-endpoint methods, using Beckman Coulter reagents (CHOL Reagent or Triglycerides GPO reagent, respectively) on SYNCHRON LX Systems. Plasma concentrations of apolipoprotein B and apolipoprotein A-I were analyzed immunonephelometrically. The determinations were done on a Dade-Behring BNII System, using antisera to either human apolipoprotein A-I (Code OUED) or apolipoprotein B (Code OSAN).

A reference curve for each apolipoprotein was generated using a standard protein serum, and validity controls were run each time the instrument was used. Plasma folate concentrations were evaluated in a clinical laboratory using the Ciba-Corning ACS Folate assay kit. Plasma homocyst(e)ine determinations were completed using the HPLC method of Jacobsen et al. Jacobsen D W, Gatautis V J, Green R, Robinson K, Savon S R, Secic M, Ji J, Otto J M, Taylor L M Jr. Rapid HPLC determination of total homocysteine and other thiols in serum and plasma: sex differences and correlation with cobalamin and folate concentrations in healthy subjects. Clin Chem 1994;40:873-81. Plasma vitamin C (a compliance marker) was measured by HPLC method, Wagner E, Lindley B, Coffin R, High performance liquid chromatography determination ascorbic acid in urine. Journal of Chromatography 1979;163:225-9, in a clinical laboratory, in a clinical laboratory.

Three-day dietary food records were obtained at baseline and during each dietary period and included one weekend day. Practical instructions on the preparation of food records were by a registered dietitian. A dietitian maintained contact with the participants at least weekly to ensure comprehension of and compliance with the dietary regimens.

The composition of orange juice consumed by participants during each dietary period included phytochemicals. During the first stage, these included monoterpenes (including 35-110 ppm limonene), limonoid terpenes (including 59-102 ppm limonoid glucoside and 38-54 ppm limonin glucoside), flavonoids including the flavanones hesperidin (20-70 ppm) and naringin (2-6 ppm) and including 0.7-2 ppm methoxyflavones. The phytochemicals during the second stage were double these. During the third stage, they were triple these values. Concerning the essential nutrients, the first dosage period had 74.9 mg vitamin C and 68.2 mg folate. The second dosage period had 149.8 mg vitamin C and 125.6 μg folate. The third dosage period had 224.7 mg vitamin C and 188.4 folate.

Statistics

Statistical analysis of the outcome measured during the four treatment periods was carried out using repeated-measures analysis of variance (ANOVA) for changes from pretreatment baseline values. ANOVA was followed by Dunnett's t-tests for comparing all treatment periods with the baseline and by Tukey's HSD test for pairwise comparison of percent changes from baseline during the experimental periods. Washout values were compared to baseline by paired t-test.

Results

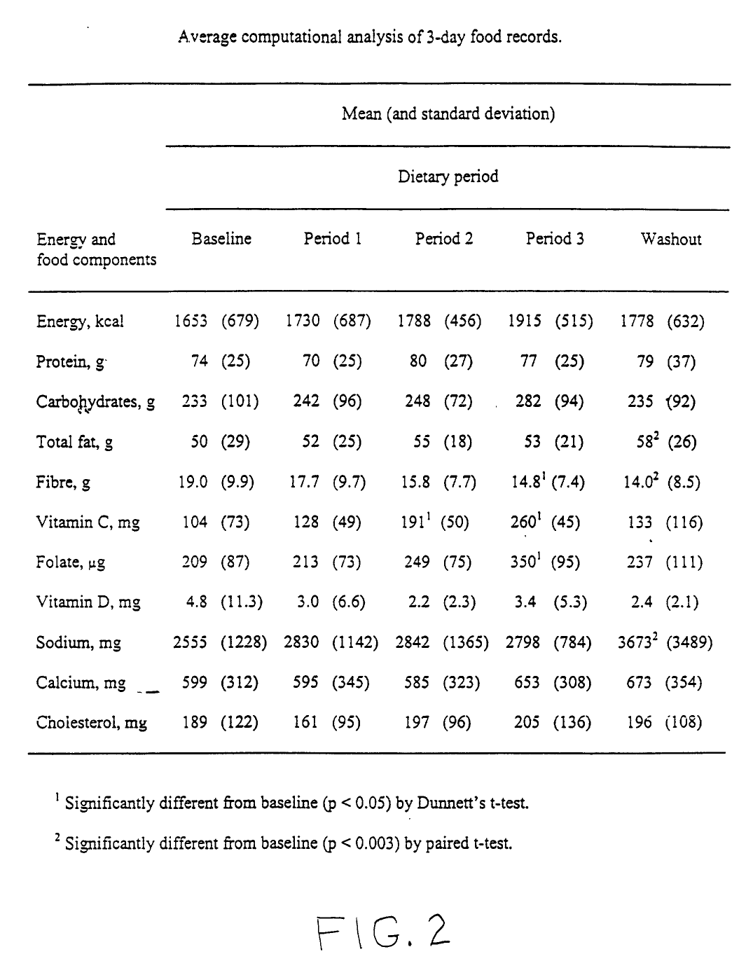

Analyses of dietary food records for baseline, dietary periods and the washout period are presented in FIG. 2. The intake of total energy, protein, carbohydrates, total fat, calcium, sodium and cholesterol was not significantly different in any of the treatment periods. The intake of fibre significantly decreased during the third period and remained significantly lower than baseline during the washout. In accordance with experimental design, vitamin C and folate intakes were significantly affected by the consumption of orange juice, with levels of vitamin C being elevated during the second and third periods and levels of folate being elevated during the third period. Intakes of both vitamin C and folate returned to baseline levels during the washout period.

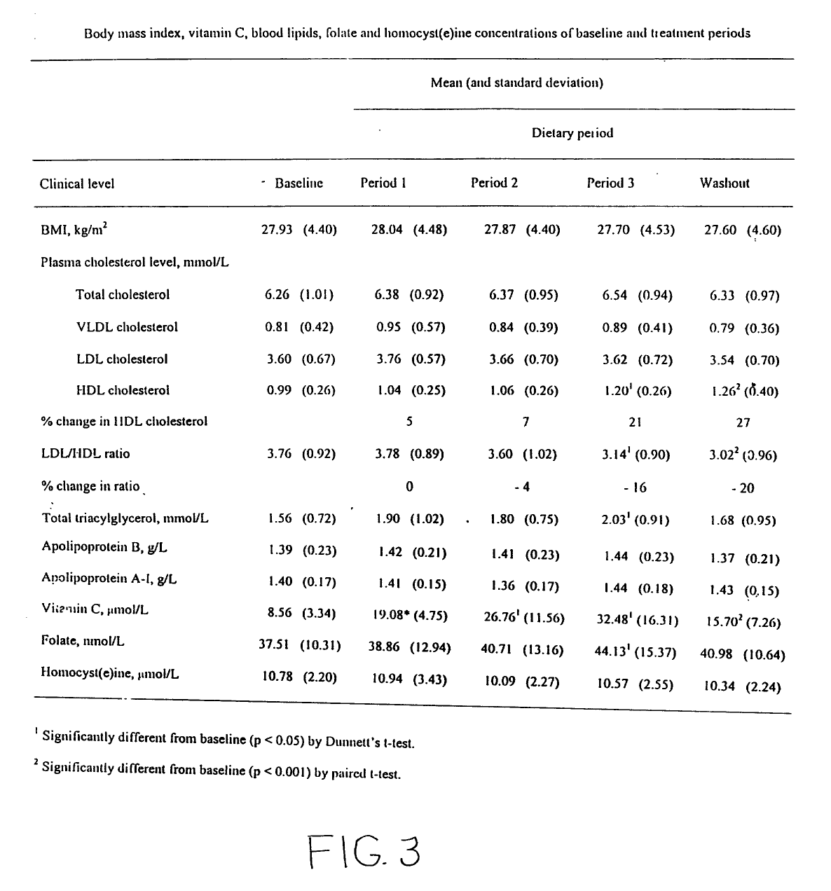

Changes in the subjects' respective baseline characteristics during the treatment with increasing doses of the orange citrus composition and during the subsequent washout period are presented in FIG. 3. The results demonstrate that the citrus composition which was administered had no significant effect on body weight (not shown in FIG. 3) and body mass index (BMI), on concentrations of apolipoprotein B and Al, or on concentrations of most of the plasma lipids. However, the treatment significantly altered the HDL cholesterol content, the ratio of LDL/HDL cholesterol concentrations increased during the third state of treatment by 21% (p≦0.001) and 30%, (p≦0.02), respectively, while the ratio of LDL/HDL cholesterol decreased by 16% (p≦0.005) during the same period. Pairwise comparison conducted for HDL cholesterol and for the ratio of LDL/HDL cholesterol revealed that for both parameters, percent changes from baseline during the third period of treatment were significantly different from the changes induced during the first or the second period (p≦0.05 for each comparison, respectively) but changes from the baseline observed during the first and the second period of treatment were not statistically different from each other (not shown).

FIG. 3 also shows that the administered citrus composition, in addition to influencing blood lipids, had a significant effect on plasma concentrations of vitamin C and folate. Plasma vitamin C concentrations were substantially elevated during all treatment periods (p≦0.001). The responses progressed in a sequential manner with increasing doses of the juice (2.3-, 3.1- and 3.8-fold increases from baseline during the first, second and third period, respectively). Plasma folate concentrations increased by 18% (p≦0.01) during the third stage of treatment. Plasma homocyst(e)ine concentrations were not significantly affected by dietary intervention as evident from FIG. 3.

During the washout period, the significantly lower ratio of LDL/HDL cholesterol, the elevated HDL cholesterol, plasma triacylglycerol and plasma vitamin C concentrations did not return to baseline values. In fact, the increases in HDL cholesterol content and the decreases in the ratio of LDL/HDL cholesterol observed during this period exceeded changes observed during the preceding third treatment period (27% increase and 20% decrease, respectively). The 1.8-fold increase in plasma vitamin C produced during the washout was less pronounced than that produced during the third period. The rise in plasma folate concentration observed during the third dietary period was partly reversed during the washout period. However, the folate washout responses were significantly higher than baseline when analyzed after log transformation (p≦0.05). Other parameters which were not influenced by the citrus composition administration were also not different from baseline during the washout period. All changes in plasma lipids, vitamin C and folate were similar in men and women participating in the study. Also, there was no tendency for these changes to be more pronounced in subjects with baseline LDL cholesterol concentrations above 4.0 mM/L.

To determine whether selected baseline parameters (the concentrations of HDL cholesterol and the ratio of LDL/HDL cholesterol) were important in producing beneficial plasma lipid responses in subjects treated, regression analysis was carried out between these parameters and changes from the baseline of plasma lipids and folate concentrations during the third period of treatment. The results revealed that changes in the ratio of LDL/HDL cholesterol induced by the third period treatment were significantly inversely related to the initial ratio of LDL/HDL cholesterol (r²=0.23, p=0.016). Similarly, changes in HDL cholesterol concentration tended to be inversely correlated with the baseline HDL cholesterol but the association was not statistically significant (p=0.059). Changes in plasma triacylglycerol and plasma folate concentrations induced by the highest dose during the third state were not significantly correlated with the initial LDL/HDL cholesterol ratio or with the initial HDL cholesterol concentration. Regression analysis was also carried out between serum folate and plasma homocyst(e)ine concentrations during the third stage of treatment. The results showed no significant relationship between both parameters when all data were included but a slight significant inverse correlation was demonstrated after exclusion of two subjects with unusually high plasma homocyst(e)ine concentrations from the analysis (r²=0.20, p=0.03).

This Example demonstrates that in a group of subjects consisting mainly of individuals with mild to moderate hypercholesterolemia, consumption of the citrus composition during the four-week period improved the plasma lipoprotein profile by significantly increasing HDL cholesterol concentrations and by reducing the ratio of LDL/HDL cholesterol. The data indicate that reduction of LDL/HDL cholesterol ratio observed during treatment was entirely due to changes in the HDL cholesterol concentrations, since LDL cholesterol concentrations were not affected by this level of intake of flavonoids and limonoids of the composition.

The increases in HDL cholesterol concentrations observed during the third stage of treatment were not associated with simultaneous increases in the principal HDL apolipoprotein, apo A-I. This suggests that the beneficial alterations in HDL cholesterol concentration induced by the citrus composition were largely due to increases in the concentration of HDL₂, a subclass of HDL containing greater proportions of cholesterol but lower proportions of apo A-I than another major HDL subclass, HDL₃. Gidez L I, Miller G J, Burstein M, Slagle S, Eder H A. Separation and quantitation of subclasses of human plasma high density lipoproteins by a simple precipitation procedure. Lipid Res 1982;23:1206-23. The increases in HDL₂ but no changes in HDL₃ in individuals with coronary heart disease. Gofman J W, Young W, Tyandy R. Ischemic heart disease, atherosclerosis, and longevity.

Circulation 1966;34:679-97.

Concerning the statistically significant elevation of plasm triacylglycerol at the third stage, this did not exceed the normal range and may not be clinically significant or result in increased cardiovascular risk. The fructose and sucrose present in the juice administered at high concentrations might contribute to the effect since both have been shown to raise plasma triacylglycerol concentrations in normo- and hyperlipidemic subjects. Hollenbeck C B. Dietary fructose effects on lipoprotein metabolism and risk for coronary artery disease. Am J Clin Nutr 1993:58(suppl):800S-809S. Truswell A S. Food carbohydrates and plasma lipids-an update. Am J Clin Nutr 1994;59(suppl):710S-718S. However, the triacylglycerol responses to dietary fructose and sucrose reported by others were often associated with a tendency of HDL cholesterol to decline (Truswell, supra) whereas in the present Example, the treatment produced increases in HDL cholesterol. This was unlikely to be the cause of the increased plasma triacylglycerol concentrations. Although the treatment composition of this Example included high intake of hesperidin, Bok et al., J Nutr, supra indicates that in hypercholesterolemic rats, dietary supplementation with a mixture of citrus flavonoids consisting largely of hesperidin did not increase serum triacylglycerol concentrations. Conversely, in Kawaguchi K, Mizuno T, Aida K, Uchino K. Hesperidin as an inhibitor of lipases from porcine pancreas and pseudomonas. Biosci Biotech Biochem 1997;61:102-4, 10% hesperidin diet significantly reduced the plasma concentration of triacylglycerols, which was attributed to ability of hesperidin to inhibit activity of lipase.

The 18% increase in plasma folate concentration induced by treatment at the third state (188.4μg/day) was relatively moderate when compared to other reported results. After similar 4-5 week periods, more pronounced (30, 50 and 100%) increases in plasma folate were reported, respectively, for individuals with coronary heart disease consuming daily 127 μg folic acid supplement, Malinow M R, Duell P B, Hess D L, Anderson P H, Druger W D, Phillipson B E, Gluckman R A, Block P C, Upson B M. Reduction of plasma homocyst(e)ine levels by breakfast cereal fortified with folic acid in patients with coronary heart disease. N Engl J Med 1998;338:1009-15, for healthy subjects consuming a vegetable and citrus-rich diet containing additional 350 μg of folate per day, Brouwer et al., J. Nutr, supra, and for women given 250 μg of folic acid supplement per day. Brouwer I A, Dusseldorp M V, Thomas C M G, Duran M, Hautvast J G A J, Eskes T K A B, Steegers-Theunissen R P M. Low-dose folic acid supplementation decreases plasma homocysteine concentrations: a randomized trial. Am J Clin Nutr 1999;69:99-104. However, other studies show that in young women, supplementation with 400 μg folic acid per day substantially increased plasma folate concentrations only after 8 weeks but not after 4 weeks. Bronstrup A, Hages M, Prinz-Langenohl R, Pietzik K. Effects of folic acid and combinations of folic acid and vitamin B-12 on plasma homocysteine concentrations in healthy, young women. Am J Clin Nutr 1998;68:1104-10.

The data of this Example indicate that changes in plasma folate concentration induced by the intake of the third stage orange juice treatment did not result in significant decreases in plasma homocyst(e)ine. However, during this period, the lowest plasma homocysteine concentrations were generally observed in subjects with the highest plasma content of folate. It is believed that lack of overall changes in homocyst(e)ine concentration was because moderate rises in plasma folate content induced by intake even at the third stage level were not sufficient to alter plasma homocyst(e)ine. Another reason for this response could be the relatively low doses of folate in this Example. Previous data suggest that a supplementation with folic acid or folate has to be 200 μg per day or greater in order to produce decreases in plasma homocyst(e)ine. Ward M, McNulty H, McPartlin J, Strain J J, Weir D G, Scott, J M. Plasma homocysteine, a risk factor for cardiovascular disease, is lowered by physiological doses of folic acid. QJM 1997;90(8):519-24.

The washout period data of this Example indicate a residual effect on plasma HDL cholesterol and LDL/HDL cholesterol ratio. The tendency towards more pronounced responses observed for plasma HDL cholesterol levels and for LDL/HDL cholesterol ratio during the washout period (27%, -20%) than during the third period (21%, -16%) generally favor an observation that these changes are due to a long term effect of flavonoids on hepatic lipoprotein metabolism. It is noted that a report regarding HepG2 cells indicated that incubation with citrus flavonoids resulted in partly irreversible reduction of apo B in cell culture medium, suggesting a long-term effect of flavonoids or their metabolites on lipoprotein metabolism in the liver. Borradaile, et al., Lipids, supra.

Analysis of correlations between the changes in selected baseline parameters induced by the treatment composition and either LDL/HDL cholesterol ratio or HDL cholesterol levels at the onset of the study of this Example showed that the beneficial alternations were generally unrelated to metabolic state of subjects during the baseline. However, a significant negative correlation was found between the baseline ratio of LDL/HDL cholesterol and the reduction of this ratio caused by the third stage treatment. Thus, these results suggest that individuals with the highest initial ratio of LDL/HDL cholesterol are most likely to experience a reduction of this ratio with the invention. An association between the baseline ratio of LDL to HDL cholesterol and a change in this ratio due to dietary intervention has also been found in a previous human trial in which hypercvholesterolemic subject were consuming diet enriched with soybean products. Kurowska E M, Jordan J, Spence J D, Wetmore S, Piche L A, Radsikowski M, Danonda P, Carroll K K. Effects of substituting dietary soybean protein and oil for milk protein and fat in subjects with hypercholesterolemia. Clin Invest Med 1997;20(3):162-70.

The present Example demonstrates that in subjects with mild to moderate hypercholesterolemia, dietary supplementation according to the invention improved the plasma lipid profile by increasing HDL cholesterol concentrations and decreasing the ratio of LDL/HDL cholesterol. The beneficial effects were still observed five weeks after termination of treatment. In addition, the Example indicates that the highest dose of dietary orange juice moderately increases plasma concentrations but does not alter plasma homocyst(e)ine.

It will be understood that the embodiments of the present invention which have been described are illustrative of some of the applications of the principles of the present invention. Numerous modifications may be made by those skilled in the art without departing from the true spirit and scope of the invention.

1. A method of increasing HDL cholesterol levels in mammals, which comprises entering into the bloodstream of a mammal an effective concentration of a treatment composition including one or more phytochemicals selected from the group consisting of a monoterpene, a terpene and a flavonoid.

2. The method in accordance with claim 1, wherein said treatment composition further includes a folate.

3. The method in accordance with claim 1, wherein said treatment composition further includes vitamin C.

4. The method in accordance with claim 1, wherein said treatment composition includes a human nutrient selected from a group consisting of a folate and vitamin C.

5. The method in accordance with claim 1, wherein said phytochemical is administered at a dosage level of at least about 100 mg per day for a plurality of days.

6. The method in accordance with claim 2, wherein said the folate is administered at a level of at least about 100 μg per day for a plurality of days.

7. The method in accordance with claim 3, wherein the vitamin C is administered at a dosage level of at least about 100 mg per day for a plurality of days.

8. The method in accordance with claim 1, wherein said monoterpene is selected from the group consisting of limonene and d-limonene.

9. The method in accordance with claim 1, wherein said terpene is a limonoid glucoside selected from the group consisting of limonin, nomilin, liminol, deoxylimonic acid, limonin carboxymethoxime, limonin-17-O-β-d-glucoside, obacunone, obacunone-17-O-β-d-glucoside, nomilin-17-O-β-d-glucoside, deacetylnomilin, deacetylnomilin-17-O-β-d-glucoside, and deacetylnomilic-17-O-β-d-glucoside.

10. The method in accordance with claim 1, wherein said flavonoid is selected from the group consisting of a flavanone and a methoxyflavone.

11. The method in accordance with claim 10, wherein said flavonone is selected from the group consisting of hesperidin, hesperetin, naringin, naringenin, narirutin, eriocitrin, didymin and poncirin.

12. The method in accordance with claim 10, wherein said methoxyflavone is selected from the group consisting of tangeretin, nobiletin, sinensetin, heptamethoxy flavone, tetra-O-methylscutellarein and hexa-O-methylgossypetin.

13. A method of decreasing the LDL/HDL serum cholesterol ratio in humans, which comprises administering to a human a treatment composition having at least two phytochemicals selected from the group consisting of a monoterpene, a terpene and a flavonoid, said treatment composition being administered at a dosage effective to decrease the LDL/HDL serum cholesterol ratio in said humans by at least 0.1.

14. The method in accordance with claim 13, wherein said treatment composition further includes a folate.

15. The method in accordance with claim 13, wherein said treatment composition further includes vitamin C.

16. The method in accordance with claim 13, wherein said phytochemical is administered at a dosage level of at least about 100 mg per day for a plurality of days.

17. The method in accordance with claim 14, wherein said the folate is administered at a level of at least about 100 μg per day for a plurality of days.

18. The method in accordance with claim 15, wherein the vitamin C is administered at a dosage level of at least about 100 mg per day for a plurality of days.

19. A method of treating a mammal having an undesirably low HDL serum cholesterol level, comprising administering to a mammal an effective concentration of a treatment composition including a phytochemical selected from a group consisting of monoterpene, a terpene and a flavonoid.

20. The treatment method in accordance with claim 19, wherein said treatment composition includes a human nutrient selected from the group consisting of a folate and vitamin C.

21. The treatment method in accordance with claim 19, wherein said phytochemical is administered at a dosage level of at least about 100 mg per day for a plurality of days.

22. The treatment method in accordance with claim 20, wherein said the folate is administered at a level of at least about 100 μg per day for a plurality of days.

23. The treatment method in accordance with claim 20, wherein the vitamin C is administered at a dosage level of at least about 200 mg per day for a plurality of days.

24. A method of treating a mammal having an undesirably high LDL/HDL serum cholesterol ratio, comprising administering to a mammal a treatment composition having a phytochemical selected from the group consisting of a monoterpene, a terpene and a flavonoid.

25. The treatment method in accordance with claim 24, wherein said treatment composition includes a human nutrient selected from a group consisting of a folate and vitamin C.

26. The treatment method in accordance with claim 24, wherein said phytochemical is administered at a dosage level of at least about 100 mg per day for a plurality of days.

27. The treatment method in accordance with claim 25, wherein said the folate is administered at a level of at least about 100 μg per day for a plurality of days.

28. The treatment method in accordance with claim 25, wherein the vitamin C is administered at a dosage level of at least about 200 mg per day for a plurality of days.

29. A product for simultaneous, separate or sequential use in treating a condition of elevated LDL to HDL serum cholesterol ratio in a mammalian subject, comprising a therapeutic amount of one or more phytochemicals selected from the group consisting of a monoterpene, a terpene and a flavonoid, said therapeutic amount being sufficiently high as to lower the elevated LDL to HDL ratio.

30. The product in accordance with claim 29, wherein said treatment composition further includes a folate.

31. The product in accordance with claim 29, wherein said treatment composition further includes vitamin C.

32. The product in accordance with claim 29, wherein said treatment composition includes a human nutrient selected from a group consisting of a folate and vitamin C.

33. The product in accordance with claim 29, wherein said phytochemical is administered at a dosage level of at least about 100 mg per day for a plurality of days.

34. The product in accordance with claim 30, wherein said the folate is administered at a level of at least about 100 μg per day for a plurality of days.

35. The product in accordance with claim 31, wherein the vitamin C is administered at a dosage level of at least about 200 mg per day for a plurality of days.

36. The product in accordance with claim 29, wherein said monoterpene is selected from the group consisting of limonene and d-limonene.

37. The product in accordance with claim 29, wherein said terpene is a limonoid selected from the group consisting of limonin, nomilin, liminol, deoxylimonic acid, limonin carboxymethoxime, limonin-17-O-β-d-glucoside, obacunone, obacunone-17-O-β-d-glucoside, nomilin-17-O-β-d-glucoside, deacetylnomilin, deacetylnomilin-17-O-β-d-glucoside, and deacetylnomilic-17-O-β-d-glucoside.

38. The product in accordance with claim 29, wherein said flavonoid is selected from the group consisting of a flavanone and a methoxyflavone.

39. The product in accordance with claim 38, wherein said flavonone is selected from the group consisting of hesperidin, hesperetin, naringin, naringenin, narirutin, eriocitrin, didymin and poncirin.

40. A composition comprising a therapeutic amount of a treatment composition including one or more phytochemicals selected from the group consisting of a monoterpene, a terpene and a flavonoid, which treatment composition increases HDL serum cholesterol levels in mammals when administered at a therapeutic amount at which the HDL serum cholesterol level is increased by greater than 0.1 mmol/L.

41. The composition in accordance with claim 40, wherein said treatment composition further includes a folate.

42. The composition in accordance with claim 40, wherein said treatment composition further includes vitamin C.

43. The composition in accordance with claim 40, wherein said treatment composition includes a human nutrient selected from a group consisting of a folate and vitamin C.

44. The composition in accordance with claim 40, wherein said phytochemical is administered at a dosage level of at least about 100 mg per day for a plurality of days.

45. The composition in accordance with claim 41, wherein said the folate is administered at a level of at least about 100 μg per day for a plurality of days.

46. The composition in accordance with claim 42, wherein the vitamin C is administered at a dosage level of at least about 200 mg per day for a plurality of days.

47. The composition in accordance with claim 40, wherein said monoterpene is selected from the group consisting of limonene and d-limonene.

48. The composition in accordance with claim 40, wherein said terpene is a limonoid selected from the group consisting of limonin, nomilin, liminol, deoxylimonic acid, limonin carboxymethoxime, limonin-17-O-β-d-glucoside, obacunone, obacunone-17-O-β-d-glucoside, nomilin-17-O-β-d-glucoside, deacetylnomilin, deacetylnomilin-17-O-β-d-glucoside, and deacetylnomilic-17-O-β-d-glucoside.

49. The composition in accordance with claim 40, wherein said flavanoid is selected from the group consisting of a flavonone and a methoxyflavone.

50. The composition in accordance with claim 49, wherein said flavonone is selected from the group consisting of hesperidin, hesperetin, naringin, naringinin, narirutin, eriocitrin, didymin and poncitrin.

51. The composition in accordance with claim 49, wherein said methoxyflavone is selected from the group consisting of tangeretin, nobiletin, sinensetin, heptamethoxy flavone, tetra-O-methylscutellarein and hexa-O-methylgossypetin.

52. The composition in accordance with claim 40, wherein said HDL level is increased by greater than 0.2 mmol/L.

53. The method in accordance with claim 13, wherein said LDL/HDL ratio is decreased by at least 0.3.

54. The method in accordance with claim 13, wherein said LDL/HDL ratio is decreased by at least 0.5.
